(19) United States
(12) Patent Application Publication
(10) Pub. No.: US 2009/0081799 A1
(43) Pub. Date: Mar. 26, 2009

(54) ANALYTE EVALUATION DEVICE AND ANALYTE EVALUATION METHOD

(75) Inventor: Michihiko AKI, Kawasaki (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: FUJITSU LIMITED, Kawasaki-shi (JP)

(21) Appl. No.: 12/276,672

(22) Filed: Nov. 24, 2008

Related U.S. Application Data
(63) Continuation of application No. PCT/JP2006/310373, filed on May 24, 2006.

(51) Int. Cl. G01N 33/00; G01N 21/64

(52) U.S. Cl. 436/86; 422/82.08; 436/172; 436/94

(57) ABSTRACT

An analyte evaluation device includes: a light irradiator for inducing fluorescence emission from an analyte; a carrier for positioning the analyte; and a fluorescence detector for receiving the fluorescence, wherein the light irradiator and the fluorescence detector are situated on mutually opposing sides of the carrier, light irradiated from the light irradiator can be passed through to a side where the fluorescence detector is located, and fluorescence emission from the analyte can be induced by the transmitted light while keeping the transmitted light from directly irradiating a fluorescence detecting element of the fluorescence detector.

### CROSS-REFERENCE TO RELATED APPLICATIONS

This application is a continuation of International Application No. PCT/JP2006/310373, filed on May 24, 2006, now pending, herein incorporated by reference.

### TECHNICAL FIELD

The following embodiments relates to technology for evaluating analytes such as proteins by monitoring fluorescence.

### BACKGROUND

Significant progress has been made lately in “genomic drug discovery,” which utilizes base sequence and gene expression information obtained by rapid advances in human genome research as well as genomic information relating to proteins and the like to efficiently carry out drug development. In genomic drug discovery, the genes associated with a disease are tracked down based on genomic information and analyzed, based on which the pathogenic mechanism is elucidated, in addition to which target molecules such as the most effective proteins for treatment are determined. The designing and synthesis of candidate compounds for drugs that are pharmaceutically active against such target molecules are then carried out. Devices that are able to readily find a target molecule such as a protein are all the more important in a process such as this.

Technology which can easily measure proteins is currently under development as the field of proteomics. A currently established method carries out measurement through the combined use of two-dimensional electrophoresis and a mass spectrograph. However, a relatively large apparatus is necessary for this purpose. Hence, there exists a need for the development of new technology for understanding the medical condition of a patient at the clinical point of care, such as in the hospital laboratory or at the patient's bedside.

One technology that is capable of analyzing proteins with greater ease is known as the micro-Total Analysis System (μ-TAS) or the Lab-on-a-chip. This is a small device composed of a glass or silicon substrate measuring several centimeters square in which micrometer-size grooves (microchannels) have been created. Chemical analyses and reactions are carried out in the device. Because liquid or gas samples are made to flow through the tiny channels (which measure from several microns to several hundreds of microns in width, and from several microns to several hundreds of microns in depth), this system provides such advantages as reducing the amount of sample and the amount of waste products and high-speed processing, in addition to which it even makes it possible to miniaturize a chemical plant. Such technology thus shows much promise in bio-related applications. Moreover, μ-TAS, which is commonly translated into Japanese as “integrated chemical analysis system” or “microchemical-biochemical analysis system,” is a miniaturized chemical analysis system in which the sensors, analytic devices and the like have been miniaturized. It is thus a device in which the functions of the equipment used in an analytical chemical laboratory have all been integrated on a chip.

Biochip technology, in particular, such as DNA chips (or DNA microarrays), has attracted notice as an effective means for gene analysis. A biochip is a device composed of a substrate of glass, silicon, plastic or the like, on the surface of which numerous differing analytes made of biopolymers such as DNA or protein are arrayed in a high density as spots. A characteristic of biochips is their ability to simplify nucleic acid and protein tests in such areas as clinical diagnosis and drug treatment {see, for example, Japanese Patent Application Laid-open No. 2001-235468 (paragraphs 0002 to 0009), and Journal of American Chemical Society, 119, pp. 8916-8920 (1997)}.

In such research and development, frequent use is made of analyte evaluation technology in which a fluorescent labeling part such as a fluorescent dye is introduced by some means onto a protein, and light irradiation causes the fluorescent labeling part to emit fluorescence. Devices (e.g., Biacore 3000, available from Biacore) which analyze protein-protein interactions by measuring the reflection angle distribution of evanescent light are marketed for such a purpose, and are contributing to the field of proteomics. In addition, evanescent microscopes (e.g., BX2WI-TIRFM, from OLYMPUS), for examining the state of substance metabolism and the state of drug delivery to individual organs by measuring a fluorescent dye introduced onto a cell surface are commercially available.

These devices are characterized by the use of total reflection and illumination optics for exciting a fluorescent dye with evanescent light that has been generated, and detecting the resulting fluorescence. That is, the sample, such as proteins or cells, is fixed on the front side or surface of a transparent substrate or a metal thin-film electrode, and excitation light impinges from the back side so as to satisfy the total reflection condition.

In an optical arrangement such as this, it is possible to place the excitation optics on the back side of the substrate and the fluorescence monitoring optics on the front side of the substrate, thus enabling a large work space to be obtained on the front side of the substrate.

However, the following problems exist with prior-art measurement devices that use evanescent light.

One problem is that the intensity of the evanescent light generated at the surface of the transparent substrate or the metal thin-film electrode falls off exponentially with distance from the surface; at a distance of about one wavelength (about several hundred nanometers), the intensity becomes very weak. For this reason, only a fluorescent dye introduced onto the cell surface is capable of excitation; it is impossible to excite a fluorescent dye introduced into the cell interior (several hundreds of microns from the substrate surface) and thereby monitor the state of substance metabolism and the state of drug penetration to various organs.

Another problem is that when the substrate is used as a metal thin-film electrode and a potential is applied to the electrode, the intensity of evanescent light generated at the surface fluctuates according to the potential applied to the electrode. As a result, use in protein detection sensors that employ the nucleotide probe method is impossible.

The nucleotide probe method is a technique which, as described in Japanese Patent Application Laid-open No. 2005-283560 (claims) and elsewhere, uses the quality, in an analyte such as an attached antibody which has been bonded to a gold electrode via an electrostatic polymer, of being attracted to the electrode by the application of an electrical potential and of separating from the electrode when application of the potential is stopped, or the reverse quality. With this technology, when a fluorescent labeling part is provided on the electrostatic polymer, it is possible by turning the electrical potential on or off to change the distance between the fluorescent labeling part and the carrier. In a case where light capable of exciting the fluorescent labeling part is present, for example, when the potential is off, the fluorescent labeling part generates fluorescence, and when the potential is on, the fluorescent labeling part is quenched. Accordingly, by having the potential sweep between the low and the high sides (Ex. −300 mV and 200 mV)a, it is possible to determine the condition at which repeated generation and quenching of fluorescence ceases (cutoff condition). This cutoff condition is a distinctive value which depends on the magnitude of, for example, the molecular weight of the analyte. By way of illustration, the cutoff condition for an antibody alone is 2 kHz, whereas the cutoff condition for the antibody having a protein bonded thereto is 500 Hz. Thus, by specifically bonding a protein to an antibody which has been bonded to an electrostatic polymer and utilizing the change in the cutoff condition to ascertain that the protein has bonded to the antibody, it is possible to discover the presence of a protein which bonds specifically to the antibody or to evaluate the amount of bonded protein from the intensity of the fluorescence. However, when evanescent light is employed, the intensity fluctuates with the electrical potential applied to the electrode, making such evaluation impossible.

One known means for resolving these problems is an optical system in which excitation light impinges from the front side of a transparent substrate or a metal thin-film electrode, and which includes fluorescence monitoring optics on the same side. However, by having the excitation optics and the fluorescence monitoring optics both occupy space on the front side, a large work space cannot be obtained. Specifically, a need arises to arrange all of the following in the same space: excitation optics, including a fiberscope for illumination and accessories; fluorescence monitoring optics, including an object lens, a fiberscope and accessories; the reference electrode, voltage-applying electrode, counterelectrode and related accessories used in a nucleotide probe method; and microchannels for handling the analytes as well as related accessories (ports, tubing, etc.). As a result, combining the nucleotide probe method and microfluidics is out of the question, making it impossible to manufacture protein detection sensors having a high sensitivity and a high reproducibility.

### SUMMARY

According to one embodiment, an analyte evaluation device includes: a light irradiator for inducing fluorescence emission from an analyte; a carrier for positioning the analyte; and a fluorescence detector for receiving the fluorescence, wherein the light irradiator and the fluorescence detector are situated on mutually opposing sides of the carrier, light irradiated from the light irradiator can be passed through to a side where the fluorescence detector is located, and fluorescence emission from the analyte can be induced by the transmitted light while keeping the transmitted light from directly irradiating a fluorescence detecting element of the fluorescence detector.

According to another embodiment, an analyte evaluation method includes: using an analyte evaluation device having a light irradiator for inducing fluorescence emission from an analyte, a carrier for positioning the analyte, and a fluorescence detector for receiving the fluorescence, wherein the light irradiator and the fluorescence detector are situated on mutually opposing sides of the carrier; passing light that has been irradiated from the light irradiator through to a side where the fluorescence detector is located; and inducing fluorescence emission from the analyte by the transmitted light while keeping the transmitted light from directly irradiating a fluorescence detecting element of the fluorescence detector.

### BRIEF DESCRIPTION OF THE DRAWINGS

FIG. 1 is a schematic side view of an analyte evaluation device according to an embodiment.

### DESCRIPTION OF THE EMBODIMENTS

Embodiments are described below in conjunction with the attached diagram and the examples appearing later in the specification. The diagram, the examples and the description provided herein are given by way of illustration and are not intended to limit the scope of the invention. It is to be understood that other embodiments may fall within the purview of the embodiments in this specification insofar as they are in keeping with the gist of the invention.

The analyte evaluation device has a light irradiator for inducing fluorescence emission from an analyte, a carrier for positioning the analyte, and a fluorescence detector for receiving the fluorescence. The device is constructed in such a way that light irradiated from the light irradiator passes through the carrier and reaches the fluorescence monitoring optics side of the fluorescence detector.

The light irradiator and the fluorescence detector are situated on mutually opposing sides of the carrier. It is preferable for the light irradiator to be on the bottom side of the carrier and for the fluorescence detector to be on the top side. However, any other arrangement is acceptable, provided the light irradiator and the fluorescence detector are disposed on mutually opposing sides of the carrier.

In this specification, “evaluation” refers to qualitatively or quantitatively determining, for example, the presence or absence of an analyte, the type of analyte, the amount of analyte, the location of the analyte, or the behavior of the analyte in response to various influences such as an electromagnetic influence, a chemical influence and a biological influence.

Because the carriers used in prior-art analyte evaluation devices cannot allow light to pass through and moreover have a complex structure, the possibility of making the carrier transparent to light did not occur to anyone. As a result, up until now, the idea of using transmitted light as the excitation light was entirely unknown.

However, taking a different approach, investigations have been conducted based on the view that, were it possible to use transmitted light as excitation light, a sufficient work space could be secured on the fluorescence monitoring optics side, which would provide various benefits. As a result, it has been found that, even in a carrier having a complex structure, by reducing the film thickness, sufficient light can be made to pass through while retaining the function of a carrier. With such an arrangement, so long as the transmitted light is kept from directly irradiating a fluorescent detecting element of the fluorescence detector, when the transmitted light excites the analyte and induces it to emit fluorescence, the fluorescence can be detected without being adversely affected by the transmitted light. Cases in which irradiation from the light irradiator generates evanescent light are also conceivable, although such cases do not pose a problem so long as this does not affect analyte evaluation. However, when the above-mentioned nucleotide probe method or the like is employed, it is preferable to be able to select a condition which does not generate evanescent light.

By doing the above, it is possible to secure a sufficient work space on the fluorescence monitoring optics side. Securing a large work space makes it possible, for example, to combine the nucleotide probe method and microchannels; specifically, by employing microchannels, the time required for protein detection can be shortened to about 1/100 the time required in the prior art. With the nucleotide probe method, the sequence of operations from the step of immobilization on the gold electrode to the step of protein detection can be continuously carried out in the microchannels. It was also discovered that the protein detection reproducibility improves and the amount of reagent required is about 1/10 that in the prior art.

Moreover, because the transmitted excitation light does not then attenuate dependent on distance from the substrate but rather is constant regardless of distance from the substrate surface, it is able to generate fluorescence even when the analyte is at a distance far from the carrier. For example, it is possible to excite a fluorescent dye that has been introduced to the interior of cells and monitor, for example, the state of substance metabolism and the state of drug penetration to various organs. Accordingly, this makes it possible to demonstrate whether a drug designed to be transported to a target organ is indeed transported, and is expected to lead to a major reduction in drug development costs.

In the case of evanescent light, when the substrate is used as a metal thin-film electrode and an electrical potential is applied to this electrode, the applied potential has the undesirable effect of causing the intensity of the evanescent light to fluctuate. However, this problem is resolved in the embodiments in this specification. The specific advantages are that, on account of the foregoing and the fact that a large work space can be obtained, the switching of nucleotide probes immobilized on electrodes arranged in an array can be monitored with a multichannel detector (e.g., a charge-coupled device, or CCD), thereby making it possible to simultaneously detect from 10 to 100 types of proteins.

Moreover, the intensity of evanescent light fluctuates with changes in the refractive index of the medium, which has made it difficult to, for example, analyze protein-protein interactions in a highly viscous solvent. However, in the embodiments in this specification, the intensity of the transmitted excitation light is not affected by changes in refractive index, making such investigations possible.

To keep from directly irradiating the fluorescence detecting element of the fluorescence detector, the angle of incidence by the transmitted light with respect to the carrier surface and the relative position of the light irradiator and the fluorescence detector are suitably selected. Here, the phrase “keep from directly irradiating the fluorescence detecting element” means that it is possible for light irradiated from the light irradiator to be reflected by another body and to irradiate the fluorescence detecting element as indirect light. Needless to say, it is desirable for the quantity of such indirect light to be small. In general, the smaller the amount of transmitted light that irradiates the fluorescence detecting element, the lower the background noise level in the requested analyte evaluations.

To enable light irradiated from the light irradiator to pass through the carrier and reach the side where the fluorescence detector is located, it is essential for the carrier to allow the light irradiated from the light irradiator to pass therethrough. Obviously, if there is an object, such as a receptacle holding the carrier, the light irradiated from the light irradiator must pass through the object in order to reach the side on which the fluorescence detector is situated after passing through the carrier, then such a receptacle must also allow the light that has been irradiated from the light irradiator to pass therethrough.

The transmittance, which is the ratio of transmitted light on the side where the fluorescence detector is situated with respect to the light irradiated from the light irradiator, while not subject to any particular limitation, is preferably at least 20% for practical reasons (metal film thickness). It is sufficient for the excitation light to have an energy density of generally 500 μW/mm². This means that, at a transmittance of from 20%, the energy of irradiated light from the light irradiator is 2.5 mW. Energy of this degree is convenient in that it enables the use of a portable solid-phase laser.

The material used for the carrier may be any selected from among materials which are capable of positioning the analyte and can be used to evaluate the desired analyte. For example, glass, ceramic, plastic or metal may be used. For the purpose of attaching proteins or the like, the use of materials which include known metals (e.g., gold, platinum, titanium) or ceramic (e.g., sapphire) is preferred. For a thiol linker, gold is especially preferred. When a biopolymer with thiols is used as the analyte or as part of the analyte, the use of gold as the carrier material enables the biopolymer to be easily be attached to the carrier.

Close attention must be paid to the metal film thickness of the material used as the carrier. For example, when the carrier is composed of a gold film provided on a sapphire plate, because gold lacks adherence to sapphire, it is necessary to adopt a measure such as first providing a layer of titanium onto the sapphire plate as a bonding layer, providing thereon a barrier layer of platinum to prevent migration of the titanium layer to the gold layer due to heating during carrier fabrication, and providing a layer of gold on the barrier layer. It is thus important to suitably set the total film thickness of these materials. For example, with an arrangement in which the thickness of the sapphire plate is 350 μm, the titanium film thickness is 5 nm, the platinum film thickness is from 5 to 10 nm, and the gold film thickness is from 20 to 25 nm, it is possible to set the above-indicated transmittance to from 20 to 30%.

This carrier may be selected in any shape, such as a receptacle or plate. In cases where the analyte is provided in a liquid state, the carrier itself may be a receptacle, part of a receptacle, or inserted within a receptacle. If the carrier is in a layer form, it may be composed of a single layer or a plurality of layers.

It is often desirable for the analyte to be attachable to the carrier because this makes it possible to pass a liquid containing the analyte through a channel and over the carrier, allowing the analyte to attach to the carrier, and thus to carry out analyte evaluation.

Here, the word “attach” may refer to any type of attachment, including physical, chemical or biological attachment. Any manner of attachment, including chemical bonding such as covalent bonding or coordination bonding, biological bonding, electrostatic bonding, physical adsorption and chemical adsorption, may be used.

It is also useful to provide the surface of the carrier with structural components (analyte-bonding parts) capable of bonding with the analyte. For example, thiols and other groups capable of bonding with nucleotides may be provided on the gold as analyte-bonding parts. Also, in cases where an electromagnetic influence is used as the subsequently described outside effect, it makes sense to use all or part of the carrier as an electrode. It is conceivable to use an electrically conductive substance itself as a carrier, or to provide a layer of a conductive substance on a glass, ceramic, plastic, metal or other surface. The electrically conductive substance is exemplified by single metals, alloys, and laminates thereof. Noble metals, of which gold is a typical example, are chemically stable and may thus be preferably used.

Any suitable known devices may be used as the light irradiator for irradiating excitation light to excite the analyte or a fluorescent labeling part of the analyte, and induce it to emit fluorescence and as the fluorescence detector for detecting fluorescence emitted by the fluorescent labeling part. However, because the light irradiator and the fluorescence detector are to be employed in a very small region, it is often advantageous to use one or more optical fibers. The optical fiber used may be one having an inside diameter of from about 1 μm to about 10 mm. While there is no limitation on the light source used as the excitation light, visible light is generally preferred.

With regard to the functions of the above-described light irradiator, fluorescence detector, carrier and the like, it is preferable for at least one factor from the group consisting of the light irradiation angle, light irradiation intensity and light irradiation surface area of the light irradiator, the fluorescence detection angle and fluorescence detection surface area of the fluorescence detector, the carrier shape, the carrier surface area, the salt concentration in the medium used, and the analyte attachment density on the carrier to be adjustable. There are cases in which, if any of these factors is adjustable, it becomes possible, in analyte evaluation, to increase the detection sensitivity or to determine the type of analyte or estimate its amount from differences in the behaviors of analytes. Moreover, it becomes easier to carry out various experiments under fluorescent monitoring.

The analytes may be suitably selected according to the object of evaluation. The analyte may itself emit fluorescence. It may also be a substance having a fluorescent labeling part. Illustrative examples include heme, which is a dye included in a protein, the amino acids tryptophan and tyrosine, and at least one selected from the group consisting of fluorescent labeling part-containing drugs, proteins, DNA, RNA, antibodies, natural or artificial single-stranded nucleotides, natural or artificial double-stranded nucleotides, aptamers, products obtained by the limited degradation of antibodies with proteases, organic compounds having an affinity for proteins, biopolymers having an affinity for proteins, complexes of the above, and any combinations thereof. Alternatively, the analyte may be a substance which includes any of the above. For example, in cases where a drug is part of another body, as with a fluorescent-labeled drug within cells, the drug may be regarded as the analyte, although, depending on the case, the body itself may be regarded as the analyte.

As used in this specification, “nucleotide” refers to any one, or mixture, from the group consisting of mononucleotides, oligonucleotides and polynucleotides. Such substances are often negatively charged. A single-stranded nucleotide or a double-stranded nucleotide may be used. Specific bonding with the analyte is possible by hybridization. Protein, DNA and nucleotide may be present in an intermingled state. Biopolymers include not only molecules from living organisms, but also molecules which originate from living organisms and have been modified, and synthesized molecules.

The term “product” used above refers here to substances obtained by the limited degradation of an antibody with a protease, and includes as well, insofar as they are in keeping with the gist of the embodiments in this specification, Fab fragments or (Fab′)₂ fragments of antibodies, fragments originating from Fab′ fragments of antibodies, and also any type of derivative thereof.

Antibodies that may be used include monoclonal immunoglobulin IgG antibodies. Fragments originating from IgG antibodies that may be used include Fab and (Fab′)₂ fragments of IgG antibodies. In addition, use may also be made of fragments originating from such Fab fragments or (Fab′)₂ fragment. Illustrative examples of substances that may be used as organic compounds having an affinity for proteins include enzyme substrate analogs such as nicotinamide adenine dinucleotide (NAD), enzyme activity inhibitors and neurotransmitter inhibitors (antagonists). Illustrative examples of biopolymers having an affinity for proteins include proteins which serve as protein substrates or catalysts, and the constituent proteins which together make up molecular complexes.

The antibody drugs mentioned above are exemplified by Actemra® (common name: tocilizumab; from Chugai Pharmaceutical Co.). Of the above, for practical reasons, those which use a protein as the analyte are important, and preferred.

The fluorescent labeling part may be one that is known to the art. Examples of fluorescent labeling parts that may be preferably used include indocarbocyanine-3 (trademark, Cy3). The method for providing the fluorescent labeling part in the analyte may be selected from among any known method. For example, use may be made of a method of introduction by a chemical reaction, or of a reaction that forms a double-stranded nucleotide from a single-stranded nucleotide. Illustrative examples of the latter include oligonucleotide chains in which a fluorescent labeling part has been introduced on the 3′ end or the 5′ end.

The analyte evaluation device according to the embodiments in this specification may be used for the purposes of qualitatively or quantitatively determining, for example, the presence or absence of an analyte, the type of analyte, the amount of analyte, the location of an analyte, and the behavior of an analyte in response to various influences such as an electromagnetic influence, a chemical influence or a biological influence.

For example, information on the absence or presence, type, amount and location of an analyte that has been taken up into cells can be obtained.

Moreover, if the analyte is provided with a fluorescent labeling part and the distance between the fluorescent labeling part and the carrier is capable of being varied by an outside action, given that the emission of fluorescence is suppressed by a quenching action when the distance between the fluorescent labeling part and the carrier is small and that the emission of fluorescence can be induced when the distance between the fluorescent labeling part and the carrier is large, it is possible to obtain information on, for example, the absence or presence of the analyte, the type of analyte and the amount of analyte.

In addition, the behavior of an analyte can be understood from the fluorescence emission and quenching behavior when an electromagnetic influence is applied. For example, an electromagnetic influence can be achieved by using the carrier as an electrode, providing a counterelectrode, and applying a potential difference between these electrodes. By applying between the carrier and the counterelectrode a potential difference having a constant value, a pulsed valve, a value which changes in a stepped manner, a value which changes periodically, or a value that is some combination of any of the above, it is possible to achieve an electromagnetic influence.

By employing these various types of potential differences, it is possible to evaluate various conditions of the analyte, such as the state of expansion or contraction, detachment from the carrier, and diffusion. Moreover, it is possible to separate and discretely evaluate analytes which detach with relative ease from the carrier and analytes which are difficult to detach.

The behavior of analytes in response to various influences such as chemical influences and biological influences may similarly be understood. The chemical influences and biological influences may be of any type, including the scission of bonds such as covalent bonds or coordination bonds that have formed, and the inhibition or imparting of ionic, hydrophobic or polar interactions.

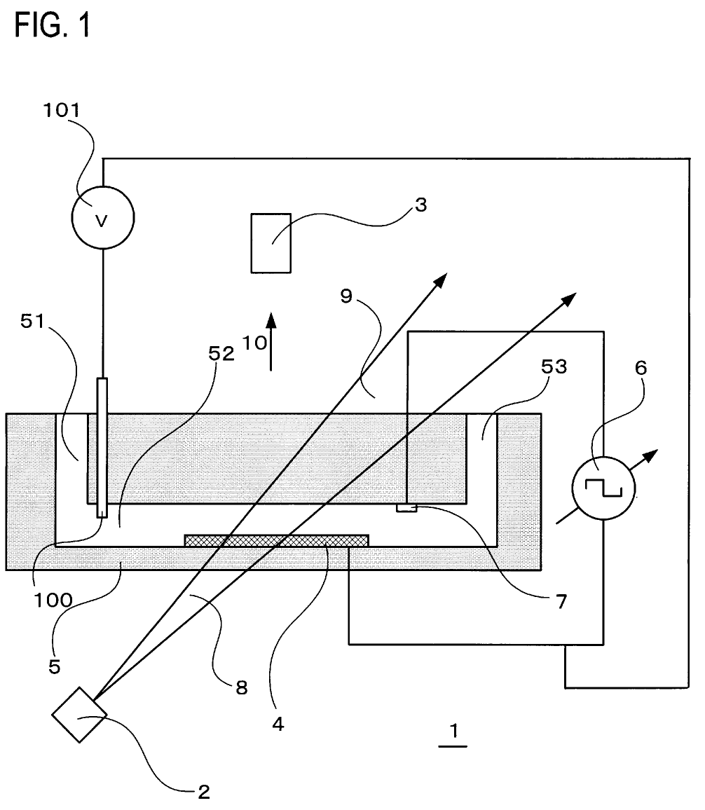

The analyte evaluation methods may be described by way of illustration in the following way in conjunction with the schematic view of an analyte evaluation device shown in FIG. 1. FIG. 1 is a schematic side view of an analyte evaluation device according to the embodiments in this specification. Referring to FIG. 1, an analyte evaluation device 1 has both a light irradiator 2 for inducing fluorescence emission from an analyte and a fluorescence detector 3, the light irradiator 2 and fluorescence detector 3 being disposed on mutually opposing sides of a carrier 4 for positioning the analyte. The analyte evaluation device 1 is provided in a microchannel 5 having an inlet 51, a flow channel 52 and an outlet 53, and an analyte (not shown) is attached thereon. The carrier 4 is an electrode, and controls a potential difference generator 6 in such a way that a desired potential difference (measured with a voltmeter 101) arises between a Ag/AgCl/3M KCl reference electrode 100 and the carrier 4.

Using such an arrangement, light 8 irradiated from the light irradiator 2 is made to pass through the carrier 4 to a side where the fluorescence detector 3 is located, and the transmitted light 9 induces the analyte to emit fluorescence 10 while being kept from directly irradiating the fluorescence detecting element of the fluorescence detector 3. A preferred embodiment of the analyte evaluation device according to the embodiments may be used in this method.

By employing the method in this way, a sufficient work space can be secured on the fluorescence monitoring optics side. Moreover, as a result, analyte evaluation can be carried out rapidly, enabling various kinds of experimentation and operations to be performed under fluorescent monitoring. In addition, the evaluation of analytes at places far removed from the carrier surface, which was impossible with conventional art utilizing evanescent light, is now possible. Moreover, even when an electrical potential is applied to the carrier, this does not have an adverse effect, making it possible to prevent the influence of changes in refractive index as in the case of technology that employs evanescent light.

### EXAMPLES

Examples and comparative examples are described below in detail.

### Example 1

Gold was used on the carrier surface in a microchannel of the structure shown in FIG. 1, and thiol groups were provided thereon as the analyte bonding part. An aqueous solution containing a nucleotide having a fluorescent labeling part on one end and having a thiol group on the other end was made to flow through this microchannel, causing the nucleotide to attach to the surface of the gold carrier.

When a potential difference of ±200 mV with respect to a Ag/AgCl/3M KCl reference electrode was applied in this state to the gold carrier and monitoring was carried out using the analyte evaluation device of FIG. 1, the repeated emission and quenching of fluorescence was observed at low frequencies, whereas only quenching occurred at frequencies of 10 kHz or more.

### Example 2

Using the same optics as in Example 1, mouse skin fibroblasts (3T3 cells) were immobilized with a cell fixative on a glass plate, covered with Parafilm to keep the cells from drying, and examined by scanning in the height direction of the cell. As a result, it was possible to observe the distribution of microtubules within the cells.

### Comparative Example 1

An attempt was made to carry out the same type of examination as in Example 2 using a microchannel like that in Example 1 except that the light irradiator 2 is disposed on the same side of the carrier 4, but this was impossible because a sufficient work space could not be secured on the fluorescence monitoring optics side.

### Comparative Example 2

Mouse skin fibroblasts (3T3 cells) were immobilized on a glass plate with a cell fixative, covered with Parafilm to keep the cells from drying, and examined with an evanescent microscope (BX2WI-TIRFM, from OLYMPUS) utilizing evanescent light. Although it was possible to observe microtubules near the glass substrate, observation of the distribution of microtubules within the cell by scanning in the height direction of the cell was not possible.

What is claimed is:

1. An analyte evaluation device, comprising: a light irradiator for inducing fluorescence emission from an analyte; a carrier for positioning the analyte; and a fluorescence detector for receiving the fluorescence, wherein
the light irradiator and the fluorescence detector are situated on mutually opposing sides of the carrier,
light irradiated from the light irradiator can be passed through to a side where the fluorescence detector is located, and
fluorescence emission from the analyte can be induced by the transmitted light while keeping the transmitted light from directly irradiating a fluorescence detecting element of the fluorescence detector.

2. The analyte evaluation device according to claim 1, wherein the analyte can be attached to the carrier.

3. The analyte evaluation device according to claim 1, wherein the carrier has a surface layer made of gold.

4. The analyte evaluation device according to claim 1, wherein the analyte has a fluorescent labeling part, and the fluorescent labeling part and the carrier are separated by a distance which can be varied by an outside action.

5. The analyte evaluation device according to claim 4, wherein the outside action is at least one selected from the group consisting of an electromagnetic influence, a chemical influence and a biological influence.

6. The analyte evaluation device according to claim 5, wherein the carrier is an electrode, and the electromagnetic influence is achieved by applying a potential difference between the carrier and a counterelectrode.

7. The analyte evaluation device according to claim 1, wherein the analyte comprises one selected from the group consisting of fluorescent labeling part-containing drugs, proteins, DNA, RNA, antibodies, natural or artificial single-stranded nucleotides, natural or artificial double-stranded nucleotides, aptamers, products obtained by the limited degradation of antibodies with proteases, organic compounds having an affinity for proteins, biopolymers having an affinity for proteins, complexes of the above, and any combinations thereof.

8. The analyte evaluation device according to claim 7, wherein the analyte comprises a protein.

9. The analyte evaluation device according to claim 1, wherein at least one factor from the group consisting of the light irradiation angle, light irradiation intensity and light irradiation surface area of the light irradiator, the fluorescence detection angle and fluorescence detection surface area of the fluorescence detector, the carrier shape, the carrier surface area, the salt concentration in a medium used, and the analyte attachment density on the carrier is adjustable.

10. The analyte evaluation device according to claim 1, wherein irradiation from the light irradiator can be carried out under a condition that does not generate evanescent light.

11. An analyte evaluation method comprising:
using an analyte evaluation device having a light irradiator for inducing fluorescence emission from an analyte, a carrier for positioning the analyte, and a fluorescence detector for receiving the fluorescence, wherein the light irradiator and the fluorescence detector are situated on mutually opposing sides of the carrier;
passing light that has been irradiated from the light irradiator through to a side where the fluorescence detector is located; and
inducing fluorescence emission from the analyte by the transmitted light while keeping the transmitted light from directly irradiating a fluorescence detecting element of the fluorescence detector.

12. The analyte evaluation method according to claim 11, wherein the analyte is attached to the carrier.

13. The analyte evaluation method according to claim 11, wherein the carrier has a surface layer made of gold.

14. The analyte evaluation method according to claim 11, wherein the analyte has a fluorescent labeling part, and the fluorescent labeling part and the carrier are separated by a distance which can be varied by an outside action.

15. The analyte evaluation method according to claim 14, wherein the outside action is at least one selected from the group consisting of an electromagnetic influence, a chemical influence and a biological influence.

16. The analyte evaluation method according to claim 15, wherein the carrier is an electrode, and the electromagnetic influence is achieved by applying a potential difference between the carrier and a counterelectrode.

17. The analyte evaluation method according to claim 11, wherein the analyte comprises one selected from the group consisting of fluorescent labeling part-containing drugs, proteins, DNA, RNA, antibodies, natural or artificial single-stranded nucleotides, natural or artificial double-stranded nucleotides, aptamers, products obtained by the limited degradation of antibodies with proteases, organic compounds having an affinity for proteins, biopolymers having an affinity for proteins, complexes of the above, and any combinations thereof.

18. The analyte evaluation method according to claim 17, wherein the analyte comprises a protein.

19. The analyte evaluation method according to claim 11, further comprising adjusting at least one factor from the group consisting of the light irradiation angle, light irradiation intensity and light irradiation surface area of the light irradiator, the fluorescence detection angle and fluorescence detection surface area of the fluorescence detector, the carrier shape, the carrier surface area, the salt concentration in a medium used, and the analyte attachment density on the carrier.

20. The analyte evaluation method according to claim 11, wherein irradiation from the light irradiator is carried out under a condition that does not generate evanescent light.
